(19) United States
(12) Patent Application Publication
(10) Pub. No.: US 2011/0178164 A1
(43) Pub. Date: Jul. 21, 2011

(54) APPLICATION ON MONOOLEIN AS A NEW HELPER LIPID IN TRANSFECTION

(76) Inventors: Maria Elisabete Cunha Dias Real Oliveira, Braga (PT); Paulo José Gomes Coutinho, Braga (PT); Olga Maria Fernandes Pereira Coutinho, Braga (PT); Andreia Ferreira Castro Gomes, Braga (PT); Margarida Paula Pedra Amorim Casal, Braga (PT); João P. N. Silva, Braga (PT)

(21) Appl. No.: 13/059,950

(22) PCT Filed: Aug. 17, 2009
(86) PCT No.: PCT/IB09/53619

§ 371 Date: Mar. 24, 2011

(30) Foreign Application Priority Data
Aug. 18, 2008 (PT) 104158

(51) Int. Cl. A61K 31/7088; C12N 15/63; C12N 5/00

(52) U.S. Cl. 514/44 R; 435/455; 435/375

(57) ABSTRACT

An application of monoolein-based cationic lipid systems can be used for the complexation, transport and transfection of genetic material into target cells. The lipid systems can be prepared through the conjugation of the neutral lipid monooleoyl-rac glycerol (Monoolein) with the cationic tensioactives derived from Dioctadecyldimethylammonium (DODAX) as, for example, Dioctadecyldimethylammonium Bromide (DODAB), Dioctadecyldimethylammonium Chloride (DODAC) and Dioctadecyldimethylammonium Phosphate (DODAP). The lipofection method can present high levels of biocompatibility and reduced acquisition costs, and is applicable for molecular biology purposes (in vitro application), as well as in the health area in gene therapy (in vivo application).

### FIELD OF INVENTION

The genotypic transformation of a cell implies a permanent hereditary change of the DNA and possibly its expression profile, which leads to fenotypic transformation. This phenomenon can be artificially obtained through the insertion of exogenous genetic material in the cell (transfection). The present invention relates to a new cationic lipid formulation (lipofection agent), through the conjugation of the neutral lipid monooleoyl-rac-glycerol (Monoolein) with the cationic tensioactives derived from Dioctadecyldimethylammonium (DODAX) as, for example, Dioctadecyldimethylammonium Bromide (DODAB), Dioctadecyldimethylammonium Chloride (DODAC) and Dioctadecyldimethylammonium Phosphate (DODAP), with application both in molecular biology (in vitro application) and in the health area in gene therapy (in vivo application).

### BACKGROUND OF THE INVENTION

To the phenotypic modification of a cell, induced by a hereditary change of the DNA and its expression profile, it is given the name of transformation. This phenomenon can be artificially obtained through the insertion of exogenous genetic material in the cell (transfection) and reveals itself of extreme importance both in the investigation field in Molecular Biology (in vitro application) and in Gene Therapy health area (in vivo application).

The several transfection methods currently available have been frequently grouped in two main categories: viral methods (recombinant viruses) and non-viral methods (complexation of DNA through lipids or polymers, co-precipitation of DNA with calcium phosphate, electroporation, gene gun, or DNA microinjection) (Maslov et al., 2002).

Table I summarizes the main advantages and disadvantages associated with these two types of transfection systems.

TABLE I
Comparative diagram the properties of viral and non-
viral transfection systems (Gregoriadis, 2007)

| Viral Systems | Non-viral Systems |
|---|---|
| (+) High transfection | (−) Medium transfection |
| efficieny | efficiency |
| (+) Transfection both in | (−) Limited transfection in |
| tumoral and primary cell | case of primary cell lines |
| lines | |
| (−) High immunogenicity | (+) Low immunogenicity and |
| | toxicity |
| (−) Mutagenic systems | (+) Biodegradable systems |
| (−) Low viral titre | (+) Possibility of |
| | controlled and orientated |
| | release |
| (−) Limited complexation | (+) Capacity to transport |
| capability | DNA of different sizes and |
| | conformations (CCC, OC, |
| | linear) |
| (−) Difficulty in case of | (+) Possibility of |
| continuous dosage | continuous and repetitive |
| | dosage |
| (−) Complexity in the | (+) Relatively low-priced |
| manufacturing process | and simple manufacturing |
| | process |

Among the non-viral methods, special relevance has been given to the lipofection method, which involves the use of cationic liposomes for the complexation and release of exogenous DNA in the cell interior. Felgner and his colleagues described for the first time in 1987 the use of lipidic molecules with positively charged groups for gene transfection in culture cell lines (Felgner et al., 1987).

Unlike pharmacological compounds that are transported within lipidic systems, plasmid DNA is not encapsulated in the liposomes interior, but remains tightly condensed due to the activity of small cationic vesicles that cover total- or partially the plasmid, originating specialized structures that became later known as lipoplexes. The formation of lipoplexes is highly dependent on the electrostatic attraction between the negative DNA phosphate backbone and the cationic lipid positive head-groups (Labat-Moleur et al., 1996; Meager, 1999).

The development of new lipids for application in gene therapy occurs essentially through the empirical testing of new compounds that have never been used before for that purpose, each one of them being analysed for a specific application. After the initial breakthrough given by Felgner and his collaborators, a first investigation phase resulted in the discovery of numerous molecules, as can be observed in several published documents: U.S. Pat. No. 5,279,833 (Rose, 1994) and U.S. Pat. No. 6,936,469 (G. deJong, S. L. Vanderbyl, 1998).

New developments continue to be made in the synthesis of new cationic lipids for transfection, as referred in the documents U.S. Pat. No. 5,651,981 (Asley et al, 1997) and WO2005033289 (R. MacDonald, L. Wang, 2005), where it is described the application of mixtures of the synthetic cationic lipids [1,2-dioleoyl-sn-glycero-3-ethylphosphocholine (EDOPC), 1,2-dilauroyl-sn-glycero-3-ethylphosphocholine (EDLPC), and 1,2-dimyristol-sn-glycero-3-ethylphosphocholine (EDMPC)] in transfection systems with no indication of presence of helper lipids. Another document [U.S. Pat. No. 7,067,697 (Xiang Gao, 2006)] presents mixtures of several cationic lipids combined with functional bioactive agents for pharmaceutical applications, being 1,2-dioleoyl-sn-glycero-3-phosphotidylethanolamine (DOPE) and cholesterol identified as helper lipids.

It has been stated that the inclusion of neutral helper lipids in the lipoplex formulation promotes an increase in DNA release and in transfection efficiency, by promoting the fusion process of larger lipoplexes with the cell membrane and by promoting the endocytosis of smaller dimensions lipoplexes (M. R. Almofti et al., 2003).

Depending on the origin and properties of the cell type used, the same lipofection system may however present very different transfection efficiencies, which has justified the permanent need to search new formulations adapted to each cell type.

Of the several documents published until now, the most frequently cited helper lipids are DOPE, cholesterol and its derivatives [U.S. Pat. No. 5,888,821 (R. Reszka, 1999) and U.S. Pat. No. 7,001,614 (N. Smyth-Templeton e G. N. Pavlakis, 2006)]. Therefore, even if the helper lipids promote, in general, the transfection process, they are one of several factors that affect transfection efficiency, like the cell type itself which is decisive for the process efficiency.

In addition, other parameters such as the lipoplex size and the structure type of the complexes may be also important for the development of a successful transfection system, because they limit its biofunctionality, biocompatibility and the toxicity. All these factors help to explain the reason why a high DNA complexation efficiency is not necessarily associated to a high transfection level.

Recently, the synthetic tensioactive Dioctadecyldimethylammonium Bromide (DODAB) (FIG. 1) was used by the authors of this patent in conjugation with a low-cost natural lipid (Monoolein) to develop vesicular systems which have efficiently demonstrated to condense salmon sperm DNA (Silva et al., 2008).

Monoolein, 1-monooleoyl-rac-glycerol (MO) (FIG. 2), is a neutral amphiphilic lipid of natural origin that owns the distinctiveness of possessing two inverted bicontinuous cubic phases, even in excess of water (<90% w/w), as can be observed by its phase diagram, represented in FIG. 3 (Lipowsky et al, 1995). The existence of non-lamellar phases, together with its low-cost value, indicates that monoolein could be a potential attractive helper lipid in lipofection systems.

The structure of the cationic lipid/DNA complexes strongly depends on the proportion between cationic lipid and DNA, as well as on the proportion between cationic lipid and helper lipid (May et al., 2004). In addition, the lipoplex formation itself depends on the liposomes preparation method and on the procedure that is used to complex the DNA by the cationic vesicles (Simões et al., 2005). It is also recognized that the order of addition of lipids and DNA has a critical effect on the physical properties and biological activity of the resulting lipoplexes (Tranchant et al., 2004). As follows, the effectiveness of monoolein in DNA complexation does not make obvious its application in lipofection.

Until now, the use of monoolein as helper lipid in lipofection systems hasn't been described, despite its low-cost value and its reduced citotoxicity, as is now proposed in the present invention.

Monoolein has only been referred as being part of formulations that encapsulate DNA in liposomes, and not by lipofection through direct DNA complexation [WO2003/057190 (2003)]. The application of monoolein as helper lipid also brings the additional advantage biocompatibility with several animal and human cell types.

The document U.S. Pat. No. 6,074,667 (Paavo Kinnunem et al; 1998) mentions the application of cationic lipids derived from sphingosines, using DOPE as first helper lipid and diacylglycerol derivatives as secondary helper lipid in KK-1 cell line. Unlike monoolein which belongs to the monoacylglycerol group, the diacylglycerols have two hydrophobic tails, being for that reason similar to the common adjuvants as DOPE.

### BRIEF DESCRIPTION OF THE FIGURES

FIG. 1—Chemical representation of Dioctadecyldimethylammonium Bromide (DODAB) molecule.

FIG. 2—Chemical representation of 1-monooleoyl-rac-glycerol (Monoolein) molecule.

FIG. 3—Phase Diagram of 1-monooleoyl-rac-glycerol (Monoolein) containing different lyotropic phases: lamellar structure (L_{α}; L_{β}; inverted hexagonal non-lamellar structure (H_{II}) and inverted cubic non-lamellar structure (Q_{II}).

FIG. 4—Citoxicity level comparison of different cationic lipid/DNA formulations in 293 T cell line, expressed as survival percentage (Lactate Dehydrogenase assay)

FIG. 5—Transfection efficiency comparison of different cationic lipid/DNA formulations in 293 T cell line, expressed as absolute absorbance at 420 nm (β-galactosidase assay).

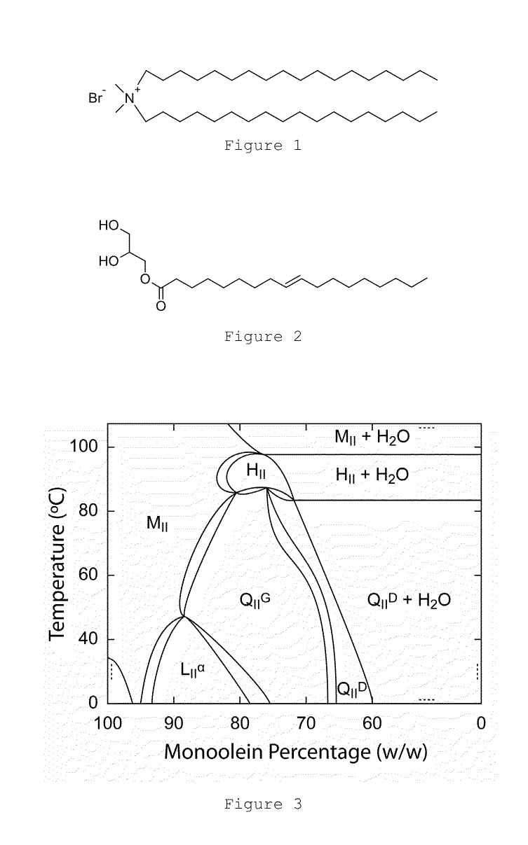

### BRIEF DESCRIPTION OF THE INVENTION

The present invention describes the application of cationic lipidic systems composed of cationic surfactants derived from Dioctadecyldimethylammonium (DODAX) and the neutral tensioactive 1-monooleoyl-rac-glycerol (Monoolein, MO). These mixtures can be used for the complexation and transport of DNA, as well as a means of genetic transformation for cells. Monoolein, 1-monooleoyl-rac-glycerol (MO), is a neutral amphiphilic lipid of natural origin that owns the distinctiveness of possessing two inverted bicontinuous cubic phases, even in excess of water (<90% w/w), as can be observed by its phase diagram, represented in FIG. 3 (Lipowsky et al, 1995). The existence of non-lamellar phases, together with its low-cost value, indicates that monoolein could be a potential attractive helper lipid in lipofection systems.

The DNA complexation, determined by the electrostatic attraction between the positively charged ammonium groups of the synthetic surfactant DODAX and the negatively charged phosphate backbone of the DNA molecules, causes the condensation of the structure thus originating highly organized cationic lipid/DNA complexes also known as lipoplexes, that also contain Monoolein in their formulation.

The inclusion of Monoolein in the lipoplex formulation grants it unique features, due to the fact that monoacylglycerols present a distinct curvature pattern from the cationic surfactant used. The decrease in structural rigidity of the DODAX vesicles, caused by the inclusion of Monoolein, increases the lateral mobility of the lipidic chain, which in the end promotes the cell membrane/lipoplex interaction, thus favouring the transfection process.

### DETAILED DESCRIPTION OF THE INVENTION

Being the cationic lipid/monoolein molar ratios applicable between the range of 0.1 to 0.9, an application example of the invention consists in the use of a lipid formulation with DODAB/Monoolein molar ratio 2:1 and charge ratios (+/−) 2.0 and 4.0, corresponding to different concentrations of the positive ammonium headgroups (DODAB) for the same concentration of negative phosphate units of the plasmid DNA.

The relative efficiency of the systems (in terms of their transfection effectiveness and citotoxicity level) was determined by direct comparison with the Lipofectamin® commercial system, in the conditions proposed by the manufacturer (Invitrogen).

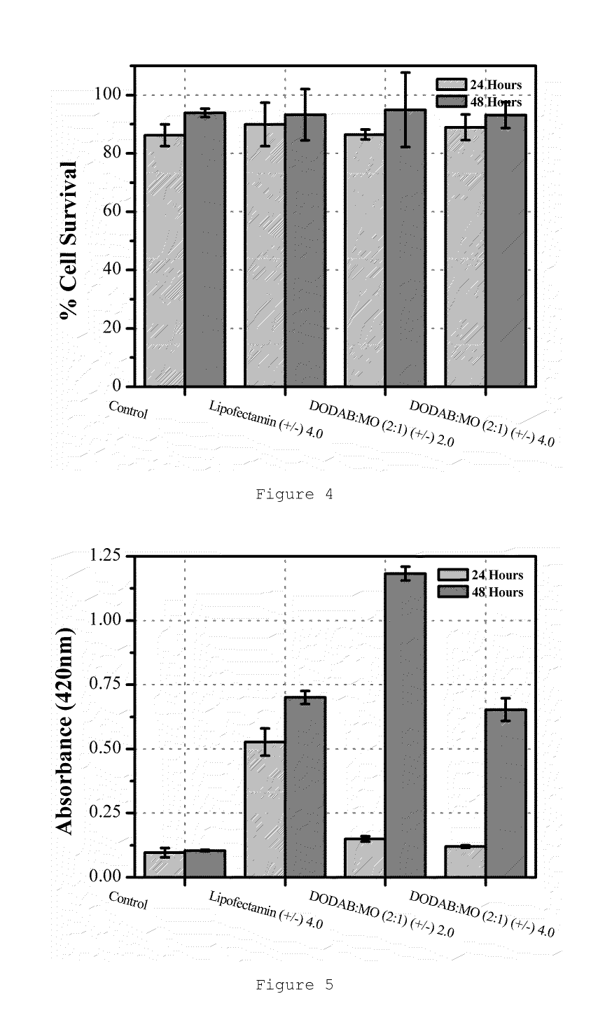

The transfection efficiency was determined through the measuring of the β-galactosidase reporter gene activity (Absorbance at 420 nm) (FIG. 4). The citotoxicity level was determined through the measuring of the Lactate Dehydrogenase activity (Absorbance at 340 nm) (FIG. 5).

In the presented example, the use of two different charge ratios (+/−) (2.0 and 4.0) for the DODAB:MO (2:1) system was made with the purpose of determining the minimum lipid concentration that could be used to obtain the maximum transfection effectiveness without citotoxicity.

For the tested formulation, although it was expected that charge ratio (+/−) 4.0 presented higher transfection efficiency than charge ratio (+/−) 2.0 because it had twice the concentration of the lipofection agent, that was not experimentally observed (FIG. 4), probably due to the citotoxicity increase at 48 h (5% to 7% cell mortality, respectively at charge ratios (+/−) 2.0 and 4.0) (FIG. 5). It must also be referred that the commercial system (Lipofectamin) presents a higher toxicity level (7% cell mortality) than the DODAB:MO (2:1) system at charge ratio (+/−) 2.0 (5% mortality).

Nevertheless, the demonstration that DODAB:MO (2:1) formulation at distinct charge ratios (+/−) present comparable transfection efficiencies (C.R. (+/−) 2.0) or even superior (C.R. (+/−) 2.0) to the commercial system, indicate that this model of invention is effective at variable concentrations of lipid and DNA.

This versatility can reveal itself useful in the application of this lipid system in other cell lines where other charge ratios (+/−) and transfection conditions are more productive to increase the process efficiency.

Preparation of Mixed Cationic Vesicles

For preparing the mixed liposome solutions, defined volumes from the stock solutions of DODAB and MO in ethanol (20 mM) were injected under vigorous vortexing to an aqueous buffer solution at 70° C. (30 mM Tris-HCl), so that the final lipid concentration ([DODAB more MO]) was 1 mM and the cationic lipid (DODAB):helper lipid (MO) molar ratio of 2:1 was obtained.

Preparation of Cationic Lipoplexes

The cationic lipoplexes were prepared through the addition of defined volumes of the mixed liposome solutions (variable concentrations of positive ammonium groups) to a constant volume of plasmid DNA solution (concentration of negative phosphate units=number of wells×1 μg DNA/well). Different volumes of Opti-MEM I Reduced Serum medium (Gibco) were used to complete a total constant volume of solution in the several formulations and charge ratios (+/−) tested (100 μL/19 mm well). The resulting lipoplexes were incubated at room temperature during 30 min without agitation.

The genetic material used was the pSV-β-gal (Invitrogen) plasmid, which was amplified by Escherichia coli DHB4 competent cell line. The extraction and purification of the plasmid DNA was made using the “Wizard® Plus Midipreps DNA Purification System” extraction kit, commercialized by Promega. The final phosphate DNA concentration (1.75 μg/μL) was measured through to absorbance at 260 nm in a Shimadzu UV-3101-PC spectrophotometer.

Cell Culture

For transfection and citotoxicity determination assays, the 293 T cell line was used and cells were cultivated in T75 cm³ flasks with DMEM medium supplemented with L-glutamin (4 mM), sodium bicarbonate (1.5 g/L), glucose (4.5 g/L), fetal bovine serum (10% v/v) and antibiotics (1 uni/ml). The cells were regularly subcultivated between passage numbers 18 and 24, being always kept at confluences lower than 90% (37° C. and 5% CO₂).

Lipofection

Twelve to sixteen hours prior to transfection, each T75 cm³ flask was washed once with PBS (1×) solution and the cells were released through the addition of trypsin solution (0.05%) (3 minutes incubation at 37° C. and 5% CO₂). The number of viable cells was quantified in a hemocytometer through the trypan blue exclusion method.

Cells were resuspended in culture medium at a density of 2×10⁵ cells/19 mm well, and were transferred to 24-Multiwell plates at a final volume of 500 μl/19 mm well (approximately 50-80% confluence twelve hours later).

In the day of transfection, the culture medium was replaced by fresh medium, and the recently prepared lipoplexes (see section “Preparation of cationic lipoplexes) were homogeneously added to each well, in a quantity that did not exceed 20% of the final volume of the solution (1 μg DNA/well). The cells were then once more incubated at 37° C. and 5% CO₂, for a 24 h-48 h period. The transfection efficiency was determined through the determination of β-galactosidase activity, the reporter gene used. The citotoxicity level was determined by measuring the activity of the Lactate Dehydrogenase.

Determination of Transfection Efficiency (β-Galactosidase Activity Assay)

In order to determine the transfection efficiency, recently incubated cells (24 h-48 h) were washed twice with PBS (1×) solution and disrupted with lysis buffer solution (RLB 1×) for 15 minutes. The cells were then recollected and centrifuged for 2 minutes at 14000 rpm to remove cellular debris. β-galactosidase enzyme activity was determined in the several samples (in arbitrary units of absorbance, a.u.) with the “β-galactosidase Enzyme Assay System with Reporter Lysis Buffer” expression kit, accordingly with the manufacturer instructions (1 μl of cellular extract+49 μl of RLB (1×) solution+50 μl of Assay (2×) buffer solution+150 μl of sodium carbonate solution, to stop the colorimetric reaction). The absorbance reading was performed at 420 nm in a 96 Multi-well plate with a SpectraMax 340PC microplate reader.

### Determination of Citotoxicity Level (Lactate Dehydrogenase Activity Assay)

For determination of the citotoxicity level in the analyzed systems, the intra and extracellular amounts of the Lactate Dehydrogenase enzyme were quantified. The collection of the culture medium in each well allowed a posterior measurement of the extracellular LDH. To obtain the value of intracellular LDH, the wells were washed with 500 μl of Tris-HCl buffer solution (15 mM), followed by mechanic scraping and ultrasound lysis of the cell samples for 3 periods of 30 seg sonication. All samples (intra and extracellular) were centrifuged for 1 minute at 13000 rpm to remove cellular debris.

The enzymatic activity of the intracellular LDH was determined [10 μl of intracellular sample+250 μl of NADH (0.31 mM) solution+10 μl of pyruvate (8.96 mM) solution to stop the colorimetric reaction]. The absorbance reading was performed at 340 nm in a 96 Multi-well plate with a SpectraMax 340PC microplate reader.

The enzymatic activity of the extracellular LDH was determined [40 μl of extracellular sample+250 μl of NADH (0.31 mM) solution+10 μl of pyruvate (8.96 mM) solution to stop the colorimetric reaction]. The absorbance reading was performed at 340 nm in a 96 Multi-well plate with a SpectraMax 340PC microplate reader.

The balance between the enzymatic activities of the intra and extracellular LDH allowed the determination of the survival and mortality percentages.

### REFERENCES

### Articles

M. A. Maslov, E. I. Syicheva, N. G. Morozova, G. A. Serebrennikova, (2002), “Cationic Amphiphiles of Both Lipid and Non-lipid Nature in Gene Therapy”, (2000), Russian Chemical Bulletin, v. 49, n. 3, pp. 385-401.
G. Gregoriadis, (2007), “Liposome Technology—II—Entrapment of Drugs and Other Materials Into Liposomes”, 3rd Edition, Editions I. Healthcare, New York (USA), pp. 424.
P. L. Felgner, T. R. Gadek, M. Holm, R. Roman, H. W. Chan, M. Wenz, J. P. Northrop, R. M. Ringold, M. Danielson, (1987), “Lipofection: a highly efficient, lipid-mediated DNA-transfection procedure, Proceedings of the National Academy of Sciences USA, n. 84, pp. 7413-7417.
F. Labat-Moleur, A. M. Steffan, C. Brisson, H. Perron, O. Feugeas, P. Furstenberger, F. Oberling, E. Brambilla, J. P. Behr, (1996), “An electron microscopy study into the mechanism of gene transfer with lipopolyamines”, Gene Therapy, n. 11, pp. 1010-1017.
A. Meager, (1999), “Gene Therapy Technologies, Applications and Regulations”, 1st Edition, Editions J.W.S. Limited, New York (USA), pp. 438.
S. W. Hui, et al., (1996), “The Role Of Helper Lipids In Cationic Liposome-Mediated Gene Transfer”, Biophysical Journal, n. 71, pp. 590-599.
P. L. Felgner, Y. I. Tsai, L. Sukhu, C. J. Wheeler, M. Manthorpe, J. Marshall, S. H. Cheng, (1995), “Improved cationic lipid formulations for in vivo gene therapy”, Ann. N.Y. Acad. Sci., n. 772, pp. 126-139.
H. Farhood, R. Bottega, R. M. Epand, L. Huang, (1992), “Effect of cationic cholesterol derivatives on gene transfer and protein kinase C activity”, Biochim. Biophys. Acta, n. 2, pp. 239-246.
G. J. Nabel, D. Gordon, D. K. Bishop, B. J. Nickoloff, Z. Y. Yang, A. Arnga, M. J. Cameron, E. G. Nabel, A. F. Chang, (1996), “Immune response in human melanoma after transfer of an allogeneic class I major histocompatibility complex gene with DNA-liposome complexes”, Proc. Natl. Acad. Sci. USA, n. 26, pp. 15388-15393.
Y. Liu, D. Liggitt, W. Zhong, C. Ti, K. Gaensler, R. Debs, (1995), “Cationic liposome mediated intravenous gene delivery”, J. Biol. Chem., n. 42, pp. 24864-24870.
R. I. Mahato, K. Kawabata, Y. Takakura, M. Hashida, (1995), “In vivo disposition characteristics of plasmid DNA complexed with cationic liposomes”, J. Drug Targeting, n. 2, pp. 149-157.
N. Oudrhiri, J. P. Vigneron, M. Peuchmaitr, T. Leclerc, J. M. Lehn, P. Lehn, (1997), “Gene transfer by guanidinium-cholesterol cationic lipids into airway epithelial cells in vitro and in vivo”, Proc. Natl. Acad. Sci. USA, n. 5, pp. 1651-1656.
P. C. A. Barreleiro, (2001), “Equilibrium and Kinetic Studies of the Binding of DNA to Cationic Liposomes”, Ph.D. thesis, Physical Chemistry Department, Center for Chemistry and Chemical Engineering, Lund University, Sweden.
J. P. Neves Silva, P. J. G. Coutinho, M.E.C.D. Real Oliveira, (2008), “Characterization of Monoolein-Based Lipoplexes Using Fluorescence Spectroscopy”, Journal of Fluorescence, pp. 1-8.
R. Lipowsky, E. Sackmann, (1995), “Structure and Dynamics of Membranes”, 2nd Edition, Elsevier Editions, New York, USA, pp. 957.
S. May, A. Ben-Shaul, (2004), “Modeling of cationic lipid-DNA complexes”, Curr. Med. Chem., n. 11, pp. 151-167.
S. Simões, A. Filipe, H. Faneca, M. Mano, N. Penacho, N. Düzgünes, M. P. Lima, (2005), “Cationic liposomes for gene delivery”, Expert Opin. Drug Deliv, n. 2, pp. 1-19.
I. Tranchant, et al., (2004), “Physicochemical Optimisation of Plasmid Delivery by Cationic Lipids”, The Journal of Gene Medicine, n. 6, pp. S24-S35.
M. R. Almofti et al., (2003), “Cationic Liposome Mediated Gene Delivery Biophysical study and Mechanism of Internalization”, Archives of Biochemistry and Biophysics, n. 410, pp. 246-253.
C. Esposito et al., (2006), “The Analysis of serum on structure, size and toxicity of DDAB-DOPE and DC-Chol-DOPE lipoplexes Contributes to Explain Their Different Transfection Efficiency”, Colloids and Surfaces B: Biointerfaces, n. 53, pp. 187-192.

### Patents

[A] J. K. Rose, (1994), “Liposomal Transfection of Nucleic Acids into Animal Cells”, U.S. Pat. No. 5,279,833, p. 1-13.
[B] G. deJong, S. L. Vanderbyl, (2005), “Methods for Delivering Nucleic Acid Molecules Into Cells and Assessment Thereof”, U.S. Pat. No. 6,936,469, p 1-39.
[C] R. MacDonald, L. Wang, (2005), “Transfection Reagents”, WO2005033289, W.I.P. Organization, p. 1-42.
[D] Xiang Gao, (2006) “Cationic Liposomes for Gene Transfer” U.S. Pat. No. 7,067,697, p. 1-44.
[E] Asley et al (1997) “Cationic Phospholipids for transfection” U.S. Pat. No. 5,651,981, p. 1-13.
[E] N. Smyth-Templeton, G. N. Pavlakis, (2006), “Liposome Complexes for Increased Systemic Delivery”, U.S. Pat. No. 7,001,614, p. 1-23.
[F] R. Reszka, (1999), “Cholesterol Derivative for Liposomal Gene Transfer”, U.S. Pat. No. 5,888,821, p. 1-4.
[H] Kinnunem et al (1998), “Liposomal Transfection Method” U.S. Pat. No. 6,074,667, p. 1-10.

1. Lipofection system, containing condensed nucleic acids, including:
a cationic lipid derived from Dioctadecyldimethylammonium (DODAX) like, for example, Dioctadecyldimethylammonium Bromide (DODAB), Dioctadecyldimethylammonium Chloride (DODAC) and Dioctadecyldimethylammonium Phosphate (DODAP); and
a neutral helper lipid as monoolein, or monoolein conjugated with cholesterol and its derivatives.

2. The lipofection system according to claim 1, including Monoolein presence in a molar fraction between 0.1 and 0.9, in proportion to the cationic lipid.

3. The lipofection system according to claim 1, including cationic lipid/nucleic acid charge ratio (+/−) being restricted between 1.0 and 4.0.

4. An application of the lipofection system as described in claim 1 comprising:
allowing transfection of mammalian cell lines with DNA and RNA of linear or circular structure, of simple or double chain.

5. An application of the lipofection system described in claim 1, wherein a procedure is made in the following manner:
the system DODAB+Monoolein is mixed with the nucleic acid, the mixture being stabilized at room temperature for a period equal or superior to 30 minutes in culture cell medium preferably with low bovine serum percentage, being afterwards added to the cells in culture.

6. An application of the lipofection system according to claim 4 designated for use in Molecular Biology and Gene Therapy areas, or other healthcare and clinical investigation areas.

7. The lipofection system according to claim 2 including cationic lipid/nucleic acid charge ratio (+/−) being restricted between 1.0 and 4.0.

8. An application of the lipofection system as described in claim 2 comprising:
allowing transfection of mammalian cell lines with DNA and RNA of linear or circular structure, of simple or double chain.

9. An application of the lipofection system as described in claim 3 comprising:
allowing transfection of mammalian cell lines with DNA and RNA of linear or circular structure, of simple or double chain.

10. An application of the lipofection system described in claim 2 wherein a procedure is made in the following manner:
the system DODAB+Monoolein is mixed with the nucleic acid, the mixture being stabilized at room temperature for a period equal or superior to 30 minutes in culture cell medium preferably with low bovine serum percentage, being afterwards added to the cells in culture.

11. An application of the lipofection system described in claim 3 wherein a procedure is made in the following manner:
the system DODAB+Monoolein is mixed with the nucleic acid, the mixture being stabilized at room temperature for a period equal or superior to 30 minutes in culture cell medium preferably with low bovine serum percentage, being afterwards added to the cells in culture.

12. An application of the lipofection system described in claim 4 wherein a procedure is made in the following manner:
the system DODAB+Monoolein is mixed with the nucleic acid, the mixture being stabilized at room temperature for a period equal or superior to 30 minutes in culture cell medium preferably with low bovine serum percentage, being afterwards added to the cells in culture.

13. An application of the lipofection system according to claim 5 designated for use in Molecular Biology and Gene Therapy areas, or other healthcare and clinical investigation areas.
